(19) United States
(12) Patent Application Publication
(10) Pub. No.: US 2004/0028623 A1
(43) Pub. Date: Feb. 12, 2004

(54) Formulations for the removal of dental plaque, tartar and dental stains

(76) Inventor: Paolo Farina, Milan (IT)

Correspondence Address: Eitan Pearl Latzer & Cohen Zedek, Suite 1001, 10 Rockefeller Plaza, New York, NY, 10020, US

(21) Appl. No.: 10/240,601

(22) PCT Filed: Apr. 3, 2001
(86) PCT No.: PCT/EP01/03757

(30) Foreign Application Priority Data
Apr. 4, 2000 (IT) MI2000A000710

(51) Int. Cl. A61K 7/28; A61K 38/05

(52) U.S. Cl. 424/50; 514/18

(57) ABSTRACT

Use of at least a substance selected from the group comprising: glutathione, pharmaceutically acceptable esters and salts of glutathione, WR-2721, sodium mercaptoethansulfonate (MESNA), diethyldithiocarbonate, sodium thiosulfate, N-acetylcystene, cytochrome P450 and cysteamine as the active substance in the preparation of pharmaceutical compositions suitable for the removal of plaque, dental calculus (tartar) and dental stain from the tooth surface, also having an anti-halitosis function, and compositions thereof.

### STATE OF THE ART

Dental plaque consists of a glycoproteic structure in which epithelial particles, mucin and food particles are dispersed, and it constitutes an ideal substrate for bacterial growth. If it is not removed, it calcifies by deposition of calcium salts of salivary origin and turns into dental calculus (tartar), a hard aggregate which is difficult to remove.

It is known that because of their bacterial component dental plaque and tartar are dangerous for tooth health and they represent the primary cause in the development of caries, gingivitis and parodontal diseases. Moreover, the presence of plaque on the tooth surface can cause halitosis because of the formation of volatile sulphurated products of the bacterial metabolism. It is therefore important both to prevent and to remove periodically plaque and dental calculus (tartar).

The most effective preventive method is an accurate oral hygiene after meals. In recent years dental research has resulted in a great improvement of instruments available for oral hygiene. For Instance, toothbrushes have been developed, which shape and geometry make it easier to clean zones of the dental arch which would be hard to reach with traditional toothbrushes (PCT/EP96/02730). In addition, toothpastes and mouthwashes containing antibacterial agents, for instance for instance chlorexidine, which are effective in reducing dental plaque and therefore tartar, have been introduced into the market.

In spite of innovations, it is still very difficult to eliminate plaque completely from all dental surfaces, especially in the interstices between teeth and in sub-gingival zones. It Is therefore still necessary to remove dental calculus (tartar) at given intervals. At present, such removal, can be carried out only mechanically by dentists or hygienists using special equipment, such as for instance ultrasounds or curettes.

This causes evident disadvantages. Apart from high costs, most patients do not regularly undergo dental examinations therefore, controlling of dental calculus (tartar) formation. This is evident from the wide spreading of parodontal diseases in the population.

It is therefore desirable the development of a simple, cheap and tolerable method directly usable by the patient enabling an effective prevention and removal of dental plaque, tartar and dental stains,

### SUMMARY OF THE INVENTION

It has now been surprisingly found that substances from the group comprising:

glutathione, pharmaceutically acceptable esters and salts of glutathione, WR-2721′, sodium mercaptoethansulfonate (MESNA), diethyldithiocarbamate, sodium thiosulfate, N-acetylcysteine, cytochrome P450 and cysteamine can dissolve dental plaque and calculus accumulation on the teeth. These substances can be used in the preparation of pharmaceutical compositions for topic use, suitable for the removal of dental plaque, tartar and dental stains from the tooth surface, also with an anti-halitosis function. Said compositions can possibly contain at least a compound selected from the group comprising an anti-inflammatory, L-ascorbic acid and a fluorinated compound.

### DETAILED DESCRIPTION OF THE INVENTION

The present invention relates to the use of at least a substance selected from the group comprising:

glutathione, pharmaceutically acceptable esters and salts of glutathione, WR-2721, sodium mercaptoethansulfonate (MESNA), diethyldithiocarbamate, sodium thiosulfate, N-acetylcysteine, cytochrome P450 and cysteamine as the active substance in the preparation of pharmaceutical compositions suitable for the removal of dental plaque, tartar and dental stains from the tooth surface, also having an anti-halitosis function.

The above-listed, substances, apart from removing plaque, inhibit the formation of bacterial catabolites, therefore having an anti-halitosis function.

Moreover, the present invention relates to pharmaceutical compositions for the removal of dental plaque, tartar and dental stains from the tooth surface, also having an anti-halitosis function, comprising as the active substance at least one of the substances elected from the group comprising:

glutathione, pharmaceutically acceptable esters and salts of glutathione, WR-2721, sodium mercaptoethansulfonate (MESNA), diethyldithiocarbamate, sodium thiosulfate, N-acetylcysteine, cytochrome P450 and cysteamine.

According to a particularly preferred application, glutathione is the active substance, preferably in an amount at least 0.05% by weight of the pharmaceutical composition, indifferently if said composition is in a solid, liquid or semiliquid form, usually at a unit dose between 0.05 and 2 g, and preferably between 0.3 and 1.5 g.

In case of single-dose solid formulations, tablets for instance, the compositions according to the invention contain an amount of glutathione per unit dose between 0.01 g and 5 g, and preferably between 0.3 g and 1.5 g. Liquid or semisolid formulations, such as toothpastes or mouthwashes, containing a percentage of glutathione between 0.05% and 20% w/w and preferably between 0.5% and 4% w/w. When present in a percentage above 20% w/w, glutathione, if left in contact with teeth, for extended periods of time, can damage their enamel. Moreover, the formulation taste becomes too disagreeable, which causes compliance problems for the patient.

The above-listed substances can be used as unique active substance or, in case of gingivitis, in association with one br more anti-inflammatories suitable for topic use. Extract of aloe pulp is preferably used as anti-inflammatory, usually in an amount corresponding to a content of active substance between 3 mg and 50 mg per unit dose, possibly in association with glycirretic acid in water soluble form, in an amount preferably between 5 mg and 100 mg per unit dose.

Single-dose solid-formulations preferably contain dry extract of aloe 200:1 in an amount corresponding to a content of active substance between 10 mg and 250 mg per unit dose, and optionally glycirretic acid in an amount preferably between 1 mg add 50 mg per unit dose. Liquid or semisolid formulations contain 0.5 to 99.5% w/w of aloe gel, and optionally 0.05% to 2% w/w of glycirretic acid.

Preferably, the pharmaceutical compositions according to the invention contain L-ascorbic acid, or one of its pharmaceutically acceptable salts of esters, which—as shown—increases anti-tartar activity of the aforesaid active substances. L-ascorbic acid is usually present in an amount between 20 mg and 200 mg per unit dose, preferably in an amount of 150 mg per unit dose or L-ascorbic acid. Is usually present in a amount between 1 mg and 50 mg per unit dose, preferably in an amount between 5 mg and 10 mg per unit dose in single-dose solid formulations, and in a percentage between 0.05 and 1% w/w in semisolid or liquid formulations. The compositions according to the invention can also contain a fluorinated compound in amounts commonly used In dental compositions. According to a preferred embodiment said fluorinated compound is sodium fluoride, which is preferably used in an amount of 0.15% w/w in liquid or semiliquid formulations, such as mouthwashes and toothpastes, and in an amount of 0.1 mg of fluoride ion in solid formulations, such as for instance mouth-dissolvable tablets.

Pharmaceutical forms suitable for carrying the compositions of the present invention are those which are commonly used for oral hygiene.

Because of the low water stability of the above-listed substances, the preferred pharmaceutical composition consists in mouthwash in single-dose bottles for immediate preparations. In such preparations the solid phase is made of the above-listed substances and possibly of other active substances which are unstable in water. Preferably, the liquid phase is fresh aloe gel 1:1. Both phases are mixed shortly before use by breaking a separation membrane.

Formulations which are particularly preferred are also tablets to dissolve in mouth or chewing gums.

The compositions of the invention can also be used in the form of gel, toothpaste or abrasive toothpaste, in which dental calculus (tartar) removal is increased by the presence of abrasive microparticles.

Another domestic method is the use of the so-called “water pic”, in which the active substance optionally in association with the above-listed substances is dissolved in the vessel containing water to be sprayed.

For professional use by dentists or hygienists, active substances can be used in pure form. For instance, they can be sprayed under pressure alone or in combination with abrasive substances. As an alternative, substances can be applied in pure form in the cavity of a dental mould prepared in advance.

As an alternative, substances can be applied, preferably in gel form, in the gap of a dental mould prepared in advance (Bite-guard). Said gel preferably contains 0.5 to 5% by weight of active substance and can also contain aloe extract and a fluorinated compound.

The invention will now be disclosed in further details with the following examples:

### EXAMPLE 1

Mouthwash in Single-Dose Bottles for Immediate Preparations:

| Solid phase | | |
|---|---|---|
| Glutathione | 600 | mg |
| Vitamin C. | 25 | mg |
| Liquid phase | | |
| Glycirretic acid, ammonium salt | 0.1% | by weight |
| Anise extract | 0.2% | by weight |
| Eugenol | 0.05% | by weight |
| Potassium: sorbate | 0.1% | by weight |
| Sodium benzoate | 0.1% | by weight |
| Vitamin C | 0.1% | by Weight |
| Aloe Barbadensis gel 1:1 | q.s. to 6 g | |
| (anthraquinones free) | | |
| Example 2 | | |
| Tablets to dissolve in mouth: | | |
| Glutathione | 600 | mg |
| Glycirretic acid, ammonium salt | 6 | mg |
| Vitamin C | 30 | mg |
| Dry extract of Aloe Barbadensis | 10 | mg p.a. |
| (anthraquinones free) | | |
| Microcrystalline cellulose | 50 | mg |
| Gelatin | 30 | mg |
| Glycerol | 0.4 | mg |
| Hydroxypropylmethylcellulose | 7 | mg |
| PEG 6000 | 0.4 | mg |
| Magnesium stearate | 4 | mg |
| Anise extract | 0.2% | by weight |
| Eugenol | 0.05% | by weight |
| Clinical test | | |

The clinical test was carried out on 10 volunteers aged between 25 and 45. Three tablets a day containing 600 mg of glutathione were administered to each volunteer, each tablet to be dissolved in mouth after a normal dental cleaning. Said tablets had the formulation described in example 2. The tartar tartar formation on the teeth of each volunteer was quantified before and during treatment, at intervals of ten days. On the tenth day of treatment a 10% reduction of dental calculus (tartar) was observed in six volunteers, and a 2% to 5% reduction in the other four volunteers. On the twentieth day of treatment a 50% reduction was observed in all volunteers. On the thirtieth day of treatment a further reduction of the content of dental calculus was observed in eight volunteers. In particular, in four of them tartar had been completely removed, even in sub-gingival and interstitial zones, whereas the other four volunteers showed a reduction of 80%. In two volunteers, because of a treatment suspension or improper administration, the amount of dental calculus (tartar) was higher than the one observed on the twentieth day.

These results show that glutathione is effective in the removal of dental calculus (tartar).

In another clinical test five volunteers were chose, all male and smokers, characterized by a strong formation of dental calculus (tartar), especially IN the zone of the lingual face of the lower incisors.

Each volunteer underwent an application of 500 mg of pure glutathione in powder on the lingual face of the lower incisors by means of a single-tuft toothbrush with medium bristles, followed by a tooth brushing for ten minutes with the same toothbrush. After a rinse with water the operation was repeated, always for ten minutes using a further 500 mg of glutathione.

All volunteers showed a complete removal of dental calculus (tartar) In the zones where brushing had been carried out. Sub-gingival and interstitial zones where brushing had not been properly carried out showed the presence of residual dental calculus (tartar).

### EXAMPLE 3

Mouthwash in Single-Dose Bottles for Immediate Preparations:

| Solid chase | | |
|---|---|---|
| Glutathione | 600 | mg |
| Vitamin C | 2 | mg |
| Liquid phase | | |
| Glycirretic acid, ammonium salt | 0.1% | by weight |
| Anise extract | 0.2% | by weight |
| Eugenol | 0.05% | by weight |
| Potassium sorbate | 0.1% | by weight |
| Sodium benzoate | 0.1% | by weight |
| Vitamin C | 0.1% | by weight |
| Sodium fluoride | 0.15% | by weight |
| Aloe Barbadensis gel 1:1 | q.s. to 6 g | |
| (anthraquinones free) | | |
| Eample 4 | | |
| Tablets to dissolve in mouth: | | |
| Glutathione | 600 | mg |
| Glycirretic acid, ammonium salt | 3 | mg |
| Vitamin C | 30 | mg |
| Dry extract of Aloe Barbadensis | 1 | mg p.a. |
| (anthraqulnones free) | | |
| Microcrystalline cellulose | 50 | mg |
| Gelatin | 30 | mg |
| Glycerol | 0.4 | mg |
| Hydroxypropylmethylcellulose | 7 | mg |
| PEG 6000 | 0.4 | mg |
| Magnesium stearate | 4 | mg |
| Sodium fluoride | equal to 0.1 mg | |
| | of ion fluoride | |
| Anise extract | 0.2% | by weight |
| Eugenol | 0.05% | by weight |

In vitro evaluation of anti-tartar activity

The in vitro evaluation of anti-tartar activity was carried out on an extracted tooth. The amount of tartar on said tooth was quantified before the test. The tooth was then immersed. Into an aqueous solution of 2% glutathlone and kept in a closed vessel without light at 30° C. Every two days said tooth was rinsed and lightly brushed and the solution was changed. The removal of tartar was vident after only four days and was completed in two to three weeks.

Clinical Test

The clinical test was carried out on 10 volunteers aged between 25 and 45. Three tablets a day containing 600 mg of glutathione were administered to each volunteer, each tablet to be dissolved in mouth after a normal dental cleaning. Said tablets had the formulation described in example 4.

Stains and tartar tartar formation on the teeth of each volunteer were quantified before and during treatment After 4 weeks of treatment a 5% reduction of dental calculus (tartar) was observed in six volunteers, and a 2% to 3% reduction in the other four volunteers. In, the sixth week of treatment a 20 to 30% reduction was observed in all volunteers. In the tenth week of treatment a further reduction of the content of dental calculus (tartar) was observed in eight volunteers. In particular, in four of them tartar had been almost completely removed, even in sub-gingival and interstitial zones, whereas the other four volunteers showed a reduction of 50%. In two volunteers, because of a treatment suspension or improper administration, the amount of dental calculus (tartar) was higher than the one observed in the sixth week. The almost complete removal of dental stains was observed in all, volunteers.

These results show that glutathione is effective in the removal of dental calculus (tartar) and dental stains.

In another clinical test five volunteers were chose, all male and smokers, characterized by a strong formation of dental calculus (tartar), especially in the zone of the lingual face of the lower incisors.

Each volunteer underwent an application of 200 mg of pure glutathione in powder on the lingual face of the lower incisors by means of a single-tuft toothbrush with medium bristles, followed-by a tooth brushing for ten minutes with the same toothbrush. After a rinse with water the operation was repeated, always for ten minutes using a further 200 mg of glutathione.

All volunteers showed a consistent reduction of dental calculus (tartar) in the zones where brushing had been carried out. Sub-gingival and interstitial zones where brushing had not been properly carried out showed the presence of residual dental calculus (tartar).

1. Use of at least a substance select d from the group consisting of: glutathione, pharmaceutically acceptable esters and salts of glutathione. WR-2721, sodium mercaptoethansulfonate (MESNA), diethyldithiocarbamate, sodium thiosulfate, cytochrome P450 and cysteamine as the active substance in the preparation of pharmaceutical compositions suitable for the removal of dental plaque and calculus from tooth surface, also having an anti-halitosis function.

2. Use according to claim 1, wherein at least a substance selected from the group consisting of: glutathione, pharmaceutically acceptable esters and salts of glutathione, WR-2721, sodium mercaptoethansulfonate (MESNA), diethyldithiocarbamate, sodium thiosulfate and cysteamine is used, as the active substance, in the preparation of pharmaceutical compositions suitable for the removal of stains from tooth surface.

3. Use according to claims 1 and 2 wherein said substance is glutathione.

4. Use according to claim 3 for the preparation of compositions in the form of single-dose solid formulations containing an amount of glutathione between 0.01 g and 5 g per unit dose.

5. Use according to claim 4, for the preparation of compositions containing an amount of glutathione between 0.05 g and 2 g per unit dose.

6. Use according to claim 5 for the preparation of compositions containing an amount of glutathione between 0.3 g and 1.5 g per unit dose.

7. Use according to claim 3 for the preparation of compositions in form of liquid or semisolid formulations containing a percentage of glutathione between 0.05% and 20% w/w.

8. Use according to claim 7 for the preparation of compositions containing a percentage of glutathione between 0.5 and 4% w/w.

9. Pharmaceutical compositions for the removal of dental plaque and tartar and stains from the tooth surface also having an anti-halitosis function, comprising as the active substance at least a substance selected from the group consisting of:
glutathione, pharmaceutically acceptable esters and salts of glutathione, WR-2721, sodium mercaptoethansulfonate (MESNA), diethyldithiocarbamate, sodium thiosulfate, N-acetylcysteine, cytochrome P450 and cysteamine, said compositions comprising an anti-inflammatory

10. Pharmaceutical compositions according to claim 9 further comprising at least a compound selected from the group consisting of: L-ascorbic acid and fluorinated compound.

11. Compositions according to claim 9, characterized in that said active substance is glutathione.

12. Compositions according to claim 11, characterized in that they are in the form of solid formulations containing glutathione in an amount between 0.01 g and 5 g per unit dose.

13. Compositions according to claim 12, characterized in that they contain glutathione in an amount between 0.05 g and 2 g per unit dose.

14. Compositions according to claim 13, characterized in that they contain glutathione in an amount between 0.3 g and 1.5 g per unit dose.

15. Compositions according to claim 11, characterized in that they are in the form is of liquid or semisolid formulations containing glutathione in a percentage between 0.05% and 20% w/w.

16. Compositions according to claim 15, characterized in that glutathione is present in a percentage between 0.5 and 4% w/w.

17. Compositions according to claim 9 characterized in that said anti-inflammatory is extract of aloe pulp.

18. Compositions according to claim 17, characterized in that they are in the form of single-dose solid formulations containing an amount of extract of aloe pulp corresponding to a content of active substance between 1 mg and 250 mg per unit dose.

19. Compositions according to claim 18, characterized in that they further contain glycirretic acid in an amount between 1 mg and 50 per unit dose.

20. Compositions according to claim 17, characterized in that they are in the form of liquid or semisolid formulations containing 0.5% to 99.6% w/w of aloe gel.

21. Compositions according to claim 20, characterized in that they further contain 0.05% to 2% w/w of glycirretic acid.

22. Compositions according to claim 10, characterized in that they are in the form of single-dose solid formulations containing L-ascorbic acid in an amount between 1 mg and 50 mg per unit dose.

23. Compositions according to claim 22, characterized in that they contain L-ascorbic acid in an amount between 5 and 10 mg per unit dose.

24. Compositions according to claim 10, characterized in that they are in the form of liquid or semisolid formulations containing L-ascorbic acid in an amount between 0.05% and 1% w/w.

25. Compositions according to claim 10 characterized in that said fluorinated compound is sodium fluoride.

26. Compositions according to claim 25, characterized in that they are in the form of liquid or semisolid formulations containing sodium fluoride in a concentration of 0.15% w/w.

27. Compositions according to claim 25, characterized in that they are in the form of solid formulations containing sodium fluoride in an amount corresponding to 0.1 mg of fluoride ions.

28. Use according to claim 3 for the preparation of compositions wherein the amount of glutathione is at least 0.05% by weight.

29. Composition according to claim 11 wherein the amount of glutathione is at least 0.05% by weight.
